(19) United States
(12) Patent Application Publication
(10) Pub. No.: US 2023/0320800 A1
(43) Pub. Date: Oct. 12, 2023

(54) Surgical Tool with Targeting Guidance

(71) Applicant: Xcelerate, Inc., Mauldin, SC (US)

(72) Inventors: Anja Glisovic, Fischen (DE); Dilantha B. Ellegala, Scottsdale, AZ (US)

(21) Appl. No.: 18/187,352

(22) Filed: Mar. 21, 2023

Related U.S. Application Data
(60) Provisional application No. 63/323,112, filed on Mar. 24, 2022.

(51) Int. Cl. A61B 34/00; A61B 90/00; A61B 34/20; A61B 34/35

(52) U.S. Cl. CPC A61B 34/70; A61B 90/361; A61B 34/20; A61B 34/35; A61B 2034/2055; A61B 2034/2065

(57) ABSTRACT

A powered surgical tool for a user to manipulate a desired tissue in surgical site within a patient may include a body having a tissue manipulation portion powerable and having a manipulation member for manipulating the desired tissue, and a projecting device with an exit port. The projecting device is powerable to emit an optical targeting marker from the exit port in a targeting direction onto the surgical site in a way that is operationally related to the operative direction and is visible to the user for providing information about at least one of a location and an orientation of the manipulation member and an operative direction within the surgical site. An imaging device may be provided to obtaining an image of the surgical site using an optical receiver attached to the body. The device may also measure and/or indicate distance to the surgical site.

### CROSS-REFERENCE TO RELATED APPLICATION

The present application is a Non-Provisional Patent Application and claims priority to U.S. Provisional Patent Application Ser. No. 63/323,112, filed Mar. 24, 2022, which is incorporated by reference herein.

### TECHNICAL FIELD

The present disclosure relates generally to surgical tools for manipulating tissue in a surgical site of a patient and relates more particularly to such surgical tools providing targeting guidance to the user.

### BACKGROUND

Powered surgical tools have been developed to manipulate (i.e., alter, cut, remove, or destroy) tissue in a desired area in a surgical site in medical or dental procedures. Such tools may use contact structures (e.g., grinders) or noncontact structures (e.g., lasers) to achieve such manipulation. Accordingly, means such as ultrasonic vibration, oscillating motion, rotary motion, laser energy, electrical energy, electromagnetic energy, magnetic energy, radiofrequency energy, radiological energy, chemical energy or reactants, mechanical energy, and thermal energy have all been employed by surgical tools to manipulate tissue to achieve a desired surgical outcome. Additional conduits, whether part of the surgical tool or part of separately inserted tools, may be used to provide fluid to irrigate and/or cool the surgical site, and/or to carry blood or removed/destroyed tissue away from the surgical site.

While performing such a surgical procedure, a surgeon needs information about position and alignment of the surgical tool as well as the tool's features and operative elements, along with information about the tool's orientation relative to the tissue in the surgical site. Such informational needs are present whether the tool is handled by the surgeon directly or whether the tool is handled robotically.

If a given surgical procedure is such that a wound cavity of the surgical site is open, the surgeon may obtain such information by direct visual observation of the surgical tool and the surgical site. If a surgical procedure is such that the targeted tissue is not directly visible to the surgeon, other methods of indirect visualization have been employed, such as cameras on other tools separate from the surgical tool, inserted into the surgical site, x-ray or magnetic resonance imaging of the patient including the surgical site, etc. The chosen method of indirect visualization varies depending on factors such as the surgical location, size and characteristics of the wound cavity, tools and technique used, surgeon preference, etc.

Indirect visualization is conventionally done using additional optical tools, such as endoscopes, which emit wide-beam illumination into the surgical site and use a camera to observe the tool and the surgical site. Conventional endoscopes provide 2-dimensional, non-stereopsis views in which the tool's alignment and/or point of contact with the tissue is conjecture. The precision of the procedure therefore relies mainly on the surgeon's ability to determine the position of the tool within the surgical site from experience and training. Also, use of a conventional endoscope allows the surgeon to observe the procedure only from the perspective of the endoscope (i.e., from the viewpoint of the location of the camera lens on the endoscope) and not from a relative “third-person” perspective (i.e., from a different viewpoint than provided by the endoscope camera lens, or from a movable viewpoint).

While existing surgical tools and methods have been successful and are satisfactory for their intended purposes, improvements in surgical tools and methods providing positioning and visualization information to a surgeon would be welcome.

### SUMMARY

According to certain aspects of the disclosure, a powered surgical tool is disclosed for a user to manipulate a desired tissue in a surgical site within a patient, the powered surgical tool may include, for example, a body including a main portion and a tissue manipulation portion, and a projecting device. The tissue manipulation portion is powerable and has a manipulation member for manipulating the desired tissue. The body and the tissue manipulation portion are configured together so as to define an operative direction of the manipulation member relative to the surgical site, wherein the tissue manipulation member is powered and aligned in the operative direction to manipulate the desired tissue disposed along the operative direction The projecting device has an exit port and is powerable to emit an optical targeting marker from the exit port in a targeting direction onto the surgical site. The exit port is located on the body portion so that the targeting direction is operationally related to the operative direction, wherein the optical targeting marker is visible to the user for providing information about at least one of a location and an orientation of the manipulation member within the surgical site and for providing information about at least one of a location and an orientation of the operative direction within the surgical site. Various options and modifications are possible.

For example, the operative direction and the targeting direction may be parallel. The body may include a central portion and a distal end extending in a distal direction from the central portion, and wherein the operative direction and the targeting direction extend in the distal direction. The body may also include a central portion and a distal end extending in a distal direction from the central portion, and wherein the operative direction and the targeting direction extend in a lateral direction angled from the distal direction, wherein the lateral direction may also be perpendicular to the distal direction. The manipulation portion may comprise, or may be at or adjacent, the body distal end of such structures.

The projecting device may include an electrically-powered light source and the exit port may include a lens for directing light from the electrically-powered light source in a beam in a targeting direction to form the optical targeting marker. An optical unit may be provided remote from the tool, the electrically-powered light source being located in the optical unit, and the optical unit may further include a camera for receiving an image obtained of the surgical site.

The exit port may emit a light beam having parallel rays so that a size of the optical targeting marker is substantially unchanged regardless of a distance between the exit port and the optical targeting marker within the surgical site. The optical targeting marker may have various shapes, such as at least one point, at least one line, characters, symbols, indicia, etc. The optical targeting marker may be sized relative to the manipulation member so as to indicate the area within the surgical site that would be contacted by the manipulation member if moved in the operative direction to contact the desired tissue. The exit port may emit a light beam having non-parallel rays centered around the targeting direction so that a size of the optical targeting marker changes depending on a distance between the exit port and the optical targeting marker within the surgical site.

The exit port may emit a light beam, and the tool may include a detector for electrooptical distancing to determine a distance between the exit port and the optical targeting marker within the surgical site. The tool may include a plurality of the exit ports and a plurality of optical receivers attached to the body for obtaining the image of the surgical site, at least one of the optical receivers used for electrooptical distancing in combination with at least one imaging device.

The projecting device may be configured to emit at least two light beams from at least one exit port, the optical targeting marker including distinct portions created by each of the at least two light beams. Two of the distinct portions may be of different wavelengths in a visible spectrum. The distinct portions may be spaced relative to the manipulation member so as to indicate the area within the surgical site that would be manipulated by the manipulation member if moved in the operative direction to contact the desired tissue. The at least one exit port may be a single exit port configured to split the light into the at least two beams. The at least one exit port may also include a plurality of exit ports, each of the exit ports being configured to emit one of the at least two beams.

At least one imaging device may be provided for obtaining an image of the surgical site. The at least one imaging device may use an optical receiver attached to the body for obtaining the image of the surgical site. The imaging device may include a camera for receiving the image from the optical receiver. The camera may be located in an optical unit remote from the tool.

The projecting device may include an electrically-powered light source and the exit port may include a lens for directing light from the electrically-powered light source in a beam in a targeting direction to form the optical targeting marker, the electrically powered light source being located in the optical unit. A plurality of the exit ports and a plurality of the optical receivers may be distributed about the tool.

The at least one imaging device may use a plurality of the optical receivers, the images obtained by the optical receivers devices being complimentary to each other, and the optical receivers may be spaced from each other on the body.

One of the imaging devices may obtain an image including the optical targeting marker along the operative direction, and another of the imaging devices may obtain an image directed differently from the operative direction. The at least one imaging device may include an optical receiver, and wherein the optical receiver may also function as the exit port of the projecting device.

Positioning markings may be distributed on the body for identifying an orientation of the body by detecting positions of the positioning markings.

A display device may be provided for displaying an image providing the information about the at least one of the orientation of and the location of the manipulation member within the surgical site and for providing the information about the at least one of the location and the orientation of the operative direction within the surgical site. The image may be a visual image of the surgical site. The image may include the optical targeting marker. The image may also include a representation of the location and the orientation of the manipulation member relative to the desired tissue. The image may be an augmented reality image.

The manipulation member may be one of a laser element, an ultrasonic element, an oscillating element, a rotary element, a thermal element, a radiofrequency element, an electrical element, an electromagnetic element, a magnetic element, a radiological element, a chemical element.

The body may include a handle and a shield, the shield being located adjacent the manipulation member of the tissue manipulation portion and configured to at least partially surround a first part of the manipulation member so that the first part is precluded from contact with the surgical site, the shield configured so that a second part of the manipulation member is exposed for use in manipulating the desired tissue within the surgical site. The exit port may be located on the shield, and at least a distal portion of the shield may be located on the body distally relative to the manipulation member, and the exit port is located on the distal portion of the shield. The projecting device may be configured with two of the exit ports, one of the exit ports being located on the shield and another of the exit ports being located on the body spaced from the shield. The device may further include an imaging device with at least two optical receivers attached to the body for obtaining the image of the surgical site, at least one of the optical receivers being located on the shield and at least another of the optical receivers being located on the body spaced from the shield.

### BRIEF DESCRIPTION OF THE DRAWINGS

FIG. 1 is an isometric view of a surgical tool according to certain aspects of the disclosure.

FIG. 2A is a closeup side view of the distal portion of the tool of FIG. 1, having one distally-oriented projecting device.

FIG. 2B is a closeup top view of the distal portion of the tool of FIG. 1.

FIG. 3 is an isometric schematic view showing a surgeon's direct visual observation of the projected optical targeting marker on the surgical site, while using the tool of FIG. 1.

FIG. 4A is a schematic view of one possible system for projecting an optical targeting marker from the tool and optionally for obtaining an image of the surgical site using an optics unit with the tool.

FIG. 4B is a schematic representation of components of the optics unit of FIG. 4a.

FIG. 5 is a schematic view showing a surgeon's visual observation on a display of the projected optical targeting marker and other optional markers on the surgical site from a 1^{st} person perspective created by projecting and imaging devices on the tool.

FIGS. 6A and 6B are diagrammatical side views, each showing potential orientations of the tool and a parallel-projected projected targeting marker with the tool moved from a first distance to a second distance (i.e., contact) relative to the surgical site.

FIGS. 7A and 7B are diagrammatical side views, each showing potential orientations of the tool and a non-parallel projected targeting marker with the tool moved from a first distance to a second distance (i.e., contact) relative to the surgical site.

FIGS. 8A and 8B are diagrammatical isometric views, each showing potential orientations of the tool and a non-linear (conical) projected targeting marker with the tool moved from a first distance to a second distance (i.e., contact) relative to the surgical site.

FIG. 9 is closeup side view of an alternate distal portion of a tool having four distally-oriented projecting devices for projecting four spaced-apart optical targeting markers.

FIG. 10 is a diagrammatic isometric view showing a tool with four projected optical targeting markers spaced equally from and around the desired point of contact using the tool having the distal portion of FIG. 9.

FIGS. 11A and 11B are diagrammatical isometric views, each showing potential orientations of the tool and four distally-oriented and parallel-projected projected targeting markers with the tool moved from a first distance to a second distance (i.e., contact) relative to the surgical site.

FIG. 11C is a diagrammatical side view of projected targeting markers including lettering or other symbolic indicia to indicate a desired point of contact.

FIG. 11D is a diagrammatical side view of projected targeting markers including lines (crossed) to indicate a desired point of contact

FIGS. 12A and 12B are diagrammatical side views, each showing potential orientations of the tool and upper and lower distally-oriented and non-parallel-projected projected targeting markers with the tool moved from a first distance to a second distance (i.e., contact) relative to the surgical site.

FIGS. 13A and 13B are diagrammatical side views, each showing potential orientations of the tool with two exit ports that emit split beams in orthogonal directions with the tool moved from a first distance to a second distance (i.e., contact) relative to the surgical site

FIG. 14A is a closeup side view of an alternate distal portion of a tool having a distal shield and four laterally-oriented and parallel-projected projecting devices for projecting four spaced-apart optical targeting markers, and having an imaging device thereon.

FIG. 14B is a closeup front view of the distal portion of the tool as in FIG. 14A.

FIG. 15 is an isometric schematic view showing a surgeon's direct visual observation of the projected optical targeting markers on the surgical site, while using the tool having the distal portion of FIG. 14A.

FIG. 16 is a schematic view showing a surgeon's visual observation on a display of the projected optical targeting markers on the surgical site, the display generated by the imaging device.

FIG. 17 is a schematic view of various system(s) that could employ the tools and related components of FIGS. 1-16.

### DETAILED DESCRIPTION

Detailed reference will now be made to the drawings in which examples embodying the present disclosure are shown. The detailed description uses numeral and letter designations to refer to features in the drawings. Like or similar reference numerals in the drawings and description have been used to refer to like or similar parts of the disclosure.

The drawings and detailed description provide a full and enabling description of the disclosure and the manner and process of making and using it. Each embodiment is provided by way of explanation of the subject matter not limitation thereof. In fact, it will be apparent to those skilled in the art that various modifications and variations may be made to the disclosed subject matter without departing from the scope or spirit of the disclosure. For instance, features illustrated or described as part of one embodiment may be used with another embodiment to yield a still further embodiment

The present disclosure is directed generally to embodiments and aspects of powered surgical tools that can be used to perform a surgical procedure on a human or animal body. Examples discussed below are exemplary only and should not be considered limiting.

The examples discussed below integrate optical features onto the surgical tool, including one or more projecting devices and/or one or more imaging devices. The disclosed projecting devices are not mere illumination devices and are configured to assist with at least one of positioning the surgical tool, visualizing, and determining and/or indicating distances between the tool and the desired (targeted) tissue for manipulation, a surgical site, and/or a wound cavity. The disclosed imaging devices are configured to assist with the visualization. Certain aspects of the examples below provide visualization with first-person perspective from the viewpoint of the tissue manipulation members of the surgical tool. Other aspects provide projection of one or more targeting markers to guide the surgeon and assist with tool alignment, as well as determining and/or representing a distance between the tool's manipulation member and the tissue. Further aspects provide predetermined alignment indicia on the surgical tool to assist in determining the surgical tool's position and orientation. The tools may be used in medical or dental surgeries or procedures on any part of the human body by a medical/dental professional or an animal body by a veterinary professional. Reference to use by a “surgeon” herein, is for convenience only, and it should be understood that such user may be any sort of medical professional, such as a physician, dentist, oral surgeon, specialist, assistant, veterinarian, etc., with the powered surgical tool being configured for the particular use by such person, either directly, cooperatively, or robotically. Thus, the powered surgical tools described and claimed herein include without limitation any sort of powered tool used by any such person in any such procedure on a human or animal body. Other aspects of the disclosure are set forth below in detail and/or are listed in the following claims.

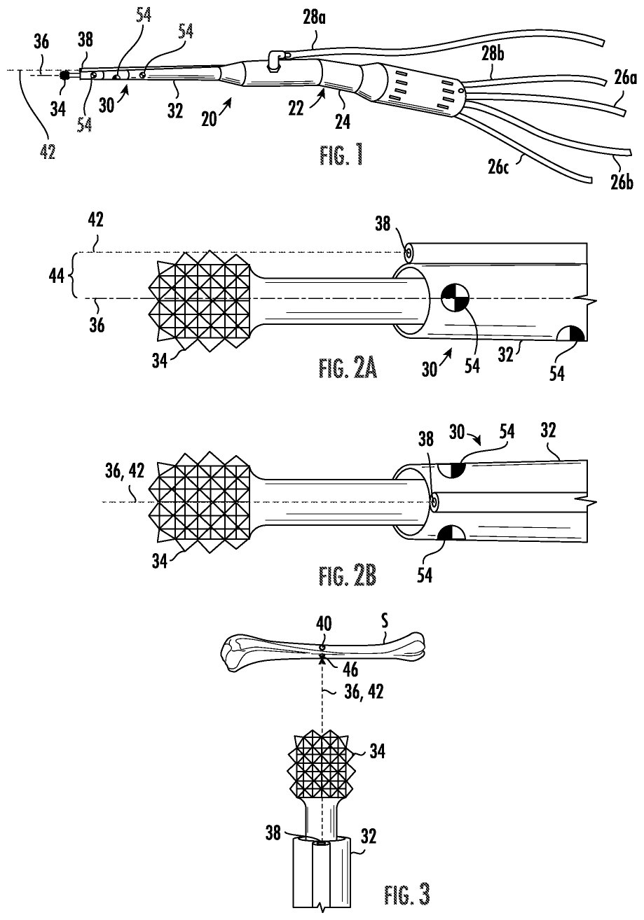

FIGS. 1-6B show a first embodiment of a powered surgical tool capable of producing an optical targeting marker for assisting the user in positioning the tool to manipulate targeted tissue, according to certain aspects of the disclosure. It should be understood that the particular disclosed aspects of the tool are exemplary only, and that many types of surgical tools could be employed with one or more of the disclosed the optical targeting features or functions based on the present disclosure. Also, the embodiment of FIGS. 1-6B includes several optional features, all of which are not required in any one particular tool according to the present disclosure to enjoy certain beneficial aspects of the present disclosure.

As shown in FIG. 1, tool 20 includes a body 22 having a central portion 24 to which one or more cables 26a,26b,26c are attached for providing respectively power, control signals, and/or optical communication function to or from the tool. One or more optional additional conduits 28a,28b may be provided for other functions, such as providing irrigation fluid to, and suction of fluid and materials from, the surgical site. The cables and conduits can be connected to one or more other components of a surgical system, as is described below. More or fewer cables or conduits can be used. For example, all or some of the cables, all or some of the conduits, or all of the cables and conduits, may if desired be within one sheath or the like for a simplified or more compact connection of the tool to the rest of the system. Also, cables carrying electronics, signaling, etc., may be replaced with wireless communication devices, if desired.

A tissue manipulation portion 30 is attached to body 22. If desired, manipulation portion 30 may be removably attachable via connecting portion 32 to central portion 24 of body. In such case, body 20 may be used with different types of manipulation members in a modular way, whether provided together as a kit or provided separately. Also, manipulation portion 30 may be removable for cleaning and reuse, or removable and disposed of in a single use embodiment.

As illustrated in FIG. 1, tissue manipulation portion 30 is attached distally to central portion 24 of body 22, with a manipulation member 34 of manipulation portion 30 extending further distally to form an end of tool 20. In the example shown in FIG. 1, manipulation portion 30 and its manipulation member 34 extend linearly along a central axis 36, although it should be understood other linear, curved, or angled shapes, whether regular or irregular, are possible. Also, manipulation member 34 need not be located at a distal end of manipulation portion 30 and/or tool 20, and more than one manipulation portion or a manipulation portion with more than one exposed section may be employed.

As illustrated, manipulation member 34 is a conventional mechanical, rotary grinder that destroys tissue by direct contact when in rotation. It should be understood that other types of manipulation members besides mechanical and/or rotary members could be employed in tool 20. Thus, as used herein, “manipulation” may be by any type of altering, cutting, removing, or destroying tissue in a desired area in a surgical site. Such tools may use contact structures (e.g., grinders, drills, ultrasound sonotrodes) or noncontact structures (e.g., lasers) to achieve such manipulation. Accordingly, besides tools employing rotary motion, tools employing oscillating motion, ultrasonic vibration, laser energy, electrical energy, electromagnetic energy, magnetic energy, radiofrequency energy, radiological energy, chemical energy or reactants, mechanical energy, and thermal energy may be utilized, with corresponding modifications or substitutions of elements in the manipulation portion, the manipulation member, the tool, and the surgical system in general. Power may be supplied by conventional electrical and/or pneumatic means. Thus, the illustration and description of an electrically-powered, mechanical, rotary manipulation portion in examples below is not intended to be limiting as to the scope of the invention.

Regardless of the type of tool and manipulation portion selected, body 22 and tissue manipulation portion 30 are configured together so as to define an operative direction of the manipulation member 34 relative to the surgical site S. For a distally located manipulation member such as member 34, the operative direction may be distal (along or parallel to central axis 36) and/or may be in a direction other than distal (at an angle to central axis 36, laterally (e.g., radially from) central axis 36, etc.), depending on the tool, the surgical procedure or a portion or step thereof, the surgeon preference, etc., As shown in FIGS. 3-6B, the operative direction is distally along axis 36. Thus, when tissue manipulation member 34 is powered and aligned in the operative direction, it can be moved along the operative direction to manipulate (i.e., contact and mechanically remove) the desired tissue disposed along the operative direction.

Tool 20 and/or its overall control system may include at least one projecting device with an exit port attached to body 22 to assist with properly positioning the tool 20 so as to contact the desired tissue (and only the desired tissue) within the surgical site. The projecting device(s) and their exit ports that can be employed with the tools disclosed herein can be of many designs, providing different functionality and benefits to the surgeon. In a most basic arrangement, the projecting device of the tools used herein can project one light beam out of one exit port to form one optical targeting marker that is essentially an illuminated “point” or “dot” where the light beam falls on the surgical site.

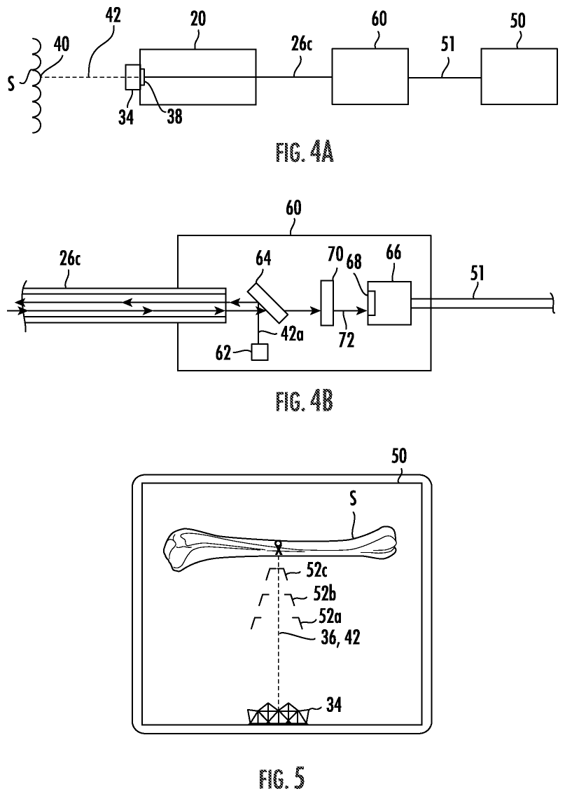

FIGS. 1-3 show such an arrangement, wherein tool 20 includes one such projecting device with an exit port 38 configured on manipulation portion 30 so as to project an optical targeting marker 40 formed by a light beam 42 from body 22 in a targeting direction onto surgical site S. Essentially, the spot where light beam 42 exiting exit port 38 impacts surgical site S is the optical targeting marker 40 (illustrated with a circle 40 in FIG. 3) so that tool alignment with respect to the desired contact point 46 can be determined and adjusted. Exit port 38 may thus comprise (only) the exit optics (lenses, apertures, slit, pinhole, masks, filters, etc.) of a projecting device for projecting a light beam, as shown in FIG. 3. However, in some embodiments, exit port 38 may also comprise an entry lens/optical receiver of an imaging system, for example, including an imaging device such as a camera with CCD for obtaining an image of the surgical site on a screen (as in FIG. 5, discussed below). Alternatively, exit port 38 may comprise the exit port for projecting the optical targeting marker, and a second lens (not shown) may be provided as the entry lens/optical receiver for obtaining the image. However, certain benefits are provided where onboard imaging is desired to have a dual-use port, lens, or equivalent, either overlapping or adjacent structure(s), that can emit the optical targeting marker and collect light of the imaging device.

FIGS. 1-2B also show optional alignment markings 54 that can be placed on tool 20 to give the surgeon visual indication of tool orientation. Markings 54 can have various shapes, such as one or more dots, lines, rings, etc., and they can be distributed along any and all sides of tool 20, including manipulation portion 30. Markings 54 may be visually observable (directly or via an on-board or separate camera) and/or they may be observable with an alternate imaging device such as x-ray imaging, MRI, rf-generated acoustic imaging, photo-acoustically generated imaging, ultrasonic imaging, or the like. Such markings can be used with any of the embodiments disclosed herein.

FIG. 4A shows a schematic view of one example of an optical system that could be employed with tool 20 to at least create a light beam for projecting at least one optical targeting marker, and FIG. 4B shows certain components of an optical unit within the optical system.

As shown in FIG. 4A, tool 20 includes an exit port 38 functionally arranged so as to project light onto and/or detect an image of surgical site S in a direction related to the orientation of manipulation member 34. The light may be generated and/or received in an optical unit 60 which is in communication with exit port 38. As shown in FIG. 4A, optical unit 60 is spaced from tool 20 and is connected by communication cable 26c which may include a waveguide such as one or more optical fibers, and/or one or more electrical power or data cable(s). Such fibers and cables may be combined into a single conduit. However, part of or all of optical unit 60 may be on or within tool 20.

Cable 26c may also include separate portions (for example a portion from exit port 38 to an optical connector within tool 20, and another portion extending from the optical connector within tool 20 to optical unit 60). Such separable portions may be useful for example, where manipulation portion 30 on which exit port 38 is mounted is removable from the rest of tool 20.

As shown in FIG. 4B, optical unit 60 is attached to and communicates with optical cable 26c and with an output cable 51 for transmitting an imaging signal to display device 50. It should be understood that transmission at least between optical unit 60 and display 50 may also be wireless.

At least one projecting device 62 is provided within optical unit 60, and may include a laser diodes or LEDs with beam-shaping lenses, which are most suitable for high intensity beam projection, preferably emitting parallel beams. In conjunction with diffractive lenses, masks, filters, slits, and apertures, laser diodes and LEDs are further suitable for patterned beam projection, as well as matrix displays/AMOLED's. A reflective device (which may as described below be a beam splitter 64) may be provided to redirect light from projecting device 62 into a waveguide such as an optical fiber (e.g., within cable 26c) toward exit port 38 to be emitted onto the surgical site. Other focusing and directing arrangements could be used, including one without reflection off a beam splitter. In combination with lenses and apertures, the projecting devices can project radially shaped beams with a focus point in a fixed direction to further aid optical distance determination, as well as indicia or lines.

As shown in FIGS. 4A and 4B, projecting device(s) 62 may employ fiber optics and wave guides so that optical unit 60 holding projecting device 62 is spaced from the tool 20. However, as stated, some laser diodes and LEDs, along with required lensing, are small enough that they may be mounted to manipulation portion 30 and or body 22, if desired. Thus, at least the light source/projecting device 62 of the optical system may be located on the tool, on the main power source of the tool nearby, or even more separately from the tool. The projecting device 62 can be a laser, a diode, a light bulb, a neon light, a matrix display, or any other light emitting source or device. Shaping of the emitted light can be achieved by utilizing apertures and lenses at or comprising element 38 at the distal exit of the wave guides in order to shape the beam as desired. Thus, the location of exit ports 38 of the projecting devices 62 identified in the drawings indicates only the point of emission of the light source, with the light to be emitted originating “upstream” from such devices 38 in one or more of the configurations noted above.

When fiber optics are used, the optical system can include not only a projecting device but also an imaging device. In this case an imaging device 66 such as a camera, for example with an imaging CCD 68, can be included in the optical system, for example within optical unit 60. Use of such an imaging device 66 allows display of an observed image on screen 50, for example, obtained through an optical receiver or lens on tool 20, which may be exit port 38 co-located at the end of the projection device or which may be another lens (not shown) on tool 20.

As shown in FIG. 4B, device 66 is arranged so that light passing into optical unit 60 though optical cable 26c passes through beam splitter 64, then though an optional safety filter 70 and potentially other optical waveguides or devices before impacting the CCD 68. Beam 42 that exits the exit port 38 to form the optical targeting marker originates from projecting device 62 as beam 42a within optical unit 60. Beam 42a is then coupled into the optical fiber of cable 26c by a conventional optical beam couple (not shown) to be projected from exit port 38 as beam 42 at the distal end of the fiber optic cable 26c. Light entering the optical receiver (e.g., exit port 38 or another port or lens on tool 20) is coupled into cable 26c and is transmitted as beam 72 within optical unit 60 to CCD 68 of optical device 66. By using fiber optics and two-way transmission, common elements may be used in the optical system for both projecting and imaging/observing, although separate pathways for projection and imaging/observing are also possible. The optical device, the CCD, and the related control equipment can be configured and programmed to provide both real time imaging and to capture video or static photos to be saved in a computer memory or removable storage media within the system or remotely.

FIG. 5 shows a resulting first-person view from the tool's perspective on screen 50. The projected optical target marker(s) 40 support the surgeon in judging distances and alignment of the tool, from this perspective. Additionally, use of a screen 50 allows display of “augmented reality” virtual markers (52a,52b,52c) on the screen which help the surgeon to determine distance and alignment in addition to displaying the projected targeting marker (42). If active distance measurement is provided, each of the virtual markers would indicate a different distance to the surgical site at desired contact point 46, so that as the displayed end of manipulation member 34 sequentially reaches each virtual marker upon approach of the tool, the surgeon gets real-time indication of the distance to contact portion 46. For example, the virtual marker distance information can be obtained via electrooptical distance determination, in which case a precise and dynamic information value can be determined and displayed, if desired, with numerical values alongside the markers as well. Alternatively, in a passive system, the markers are shown at a fixed distance from the tool head and to give a more approximate indication of distance (similar to some displays on conventional automobile rear-view cameras). Whether actively or passively based, the distance information used for plotting the virtual markers on screen 50 can be derived beforehand from the imaging device's field of view and can therefore provide indicia to guide the eye of the surgeon to help to determine the relative distance.

As illustrated, projecting device exit port 38 is located on the body so that the light originally generated by the projecting device 62 is emitted in a targeting direction operationally related to the operative direction. For example, the projecting device may project a beam 42 that, when incident on the surgical site S, creates an optical targeting marker 40 (marked with a circle in the figures for clarity, but which may be one or more points, lines, characters, geometric shapes, or any desired visible indicia). The location of optical targeting marker 40 has some predetermined relationship to the orientation and/or position of manipulation member 34 of tool 20 due to the fixed location of the exit port 38 on the tool. By knowing the predetermined relationship, the surgeon thereby obtains information about the position and orientation of manipulation member relative to surgical site S.

For example, if desired, beam 42 may extend distally parallel to and spaced at a predetermined and known distance 44 (e.g., 1 cm) from central axis 36. Thus, optical targeting marker 40 would be located distance 44 from a location 46 (the center of which is marked with an X in the figures for clarity) where central axis 36 would contact surgical site S if tool 20 were moved in an operative direction 48 (see FIG. 6B) parallel to the central axis into contact with the surgical site at point 46. In other words, beam 42 creates a visually discernable optical targeting marker 40 on surgical site S at a known distance 44 from the point of contact with the center of manipulation member 34 that will act on the surgical site if moved axially. As the surgeon moves the tool closer to the patient's body within the surgical site, optical targeting marker 40 remains visible and provides information to the surgeon so that the tool may be appropriately and reliably handled to reach and contact the desired target tissue at point 46.

The distance 44 (e.g., 1 cm, but others are suitable) is predetermined and would be made known to the surgeon during training. Thus, the skilled surgeon would understand that by first orienting tool 20 with optical targeting marker 40 on the surgical site S distance 44 (1 cm) above the desired contact location 46 (see FIG. 6A) and then moving tool 20 in the operative direction 48 (distally and axially, see FIG. 6B) toward the desired contact location while maintaining the location of the optical targeting marker, the desired contact location 46 will be contacted by the center of manipulation member 34 (along central axis 36 in this example). The distance 44 may be selected through arrangement and configuration of manipulation member 34, exit port 38, manipulation portion 30, etc. By using a sufficient offset to create beam 42 parallel to central axis 36 but spaced enough from other parts of tool 20, shadowing or blockage of the beam is prevented and optical targeting marker 40 may be made visible near to the target site 46, at distance 44.

Optical targeting marker 40 may thus be noted by direct visual observation. However, the marker and surrounding site may also be captured by an imaging device such as a camera, endoscope, or the like (separate from tool 20 or onboard with tool 20) and displayed on a display device such as an electronic screen. Therefore, optical targeting marker 40 provides information about at least one of a location and an orientation of manipulation member 34 within the surgical site and/or information about at least one of a location and an orientation of the operative direction within the surgical site.

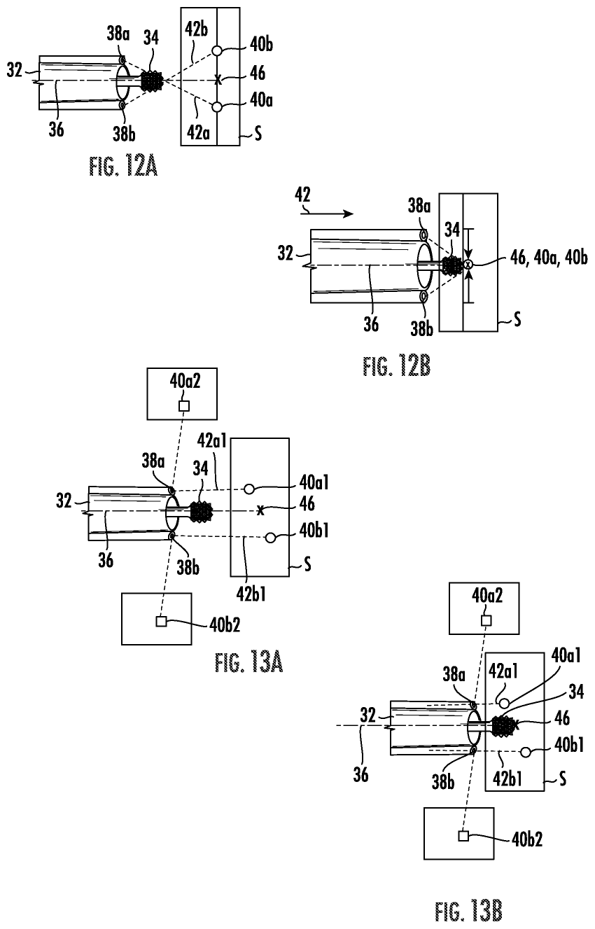

In the non-limiting example above, tool 20 uses a distal operative direction 48 (along central axis 36) and a parallel beam 42 (see FIGS. 6A and 6B). However, the operative direction 48 of contact need not be distal or parallel to central axis 36. If so, projecting device exit port 38 could be configured and aimed accordingly to project beam 42 in a complimentary direction to create optical targeting marker 40 in a desired location. For example, if the operative direction 48 is perpendicular to (extending radially outward from) central axis 36, beam 42 may extend radially outward from and aligned with the central axis, or may extend parallel to a line extending radially outward from and aligned with the central axis (see FIGS. 13A-16). Other angles and orientations are possible other than distal or radial/perpendicular, depending on factors such as the tool, the type of manipulation portion included, the surgical procedure, surgeon preference, etc.

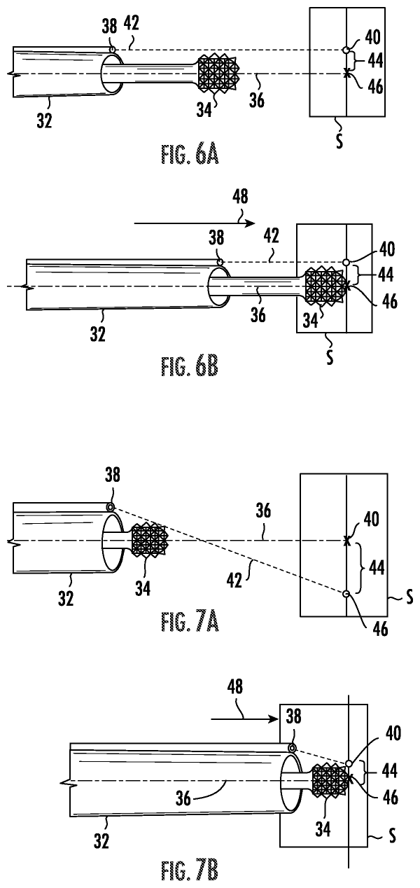

Also, beam 42 need not be projected parallel to the operative direction 48 or central axis 36. FIGS. 7A and 7B show an alternative aiming of projecting device exit port 38 and beam 42. Here, beam 42 is angled downward (as illustrated) and crosses below central axis 36. In this orientation, the location of optical targeting marker 40 on surgical site S changes as tool 20 is moved in operative direction 48. Thus, in FIG. 7A, distance 44 is greater than in FIG. 7B (when contact with desired location 46 occurs). As shown, optical targeting marker 40 is slightly above location 46 at contact in FIG. 7B, the configuration of tool 20 and projecting device exit port 38 could be such that the marker is above, below, aside, or coincident with (if shadowing is avoided) target location 46. Although operative direction 48 in FIG. 7B is shown as parallel to central axis 36, it need not be, and could instead be parallel to beam 42 or nonaligned with either, depending on factors such as tool type and configuration, surgical procedure, surgical preference, etc.

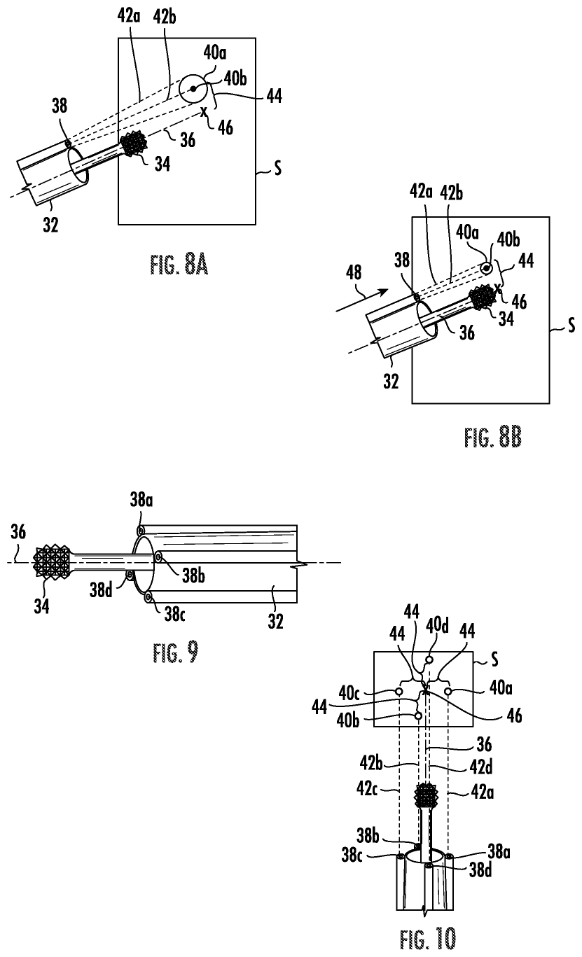

Further, the beam emitted need not be a single beam, need not project as only a point, and need not project a static-sized optical targeting marker using only parallel beams of light. FIGS. 8A and 8B show alternate optical targeting marker(s) comprising an outer (circular) section 40a formed by beam 42a and central (point) section 40b formed by beam 42b. Beam 42a is conical, and beam 42b is linear as with beam 42 above. Beam shaping may be created by masks, apertures, filters, or lenses at one or more exit port(s) 38. Due to the conical shape of beam 42a, outer section 40a has a first size at a first distance from surgical site S (FIG. 8A) and a second (smaller) size at a second smaller distance, for example when moved to bring manipulation member 34 into contact with desired location 46 (FIG. 8B). Central section 40b is optional, and could be, as illustrated, aligned similarly to beam 42 in FIGS. 6A and 6B, at distance 44 from contact location 46. Use of non-parallel, non-axial light beams causes the size of optical targeting marker 40a to change size as tool is moved closer to or further from the surgical site, the observed size giving the surgeon information as to the distance to the surgical site. As above, it would also be possible to arrange optical targeting marker 40a so as to be non-parallel to central axis 36 and/or operative direction 48, depending on the factors noted above.

It should be understood that in embodiments where multiple beams are being projected (whether from a single exit port or multiple exit ports), different beams may be made of different wavelengths of light, which can also provide information to a surgeon. Such beams can be created in different ways, for example, coupling into an optical fiber or waveguide two colors of light or a broad spectrum of light, then separating the colors using a wavelength-sensitive filter or beam splitter, or by using polarization of light and polarized filtering. Such color-differentiation can be applied to all embodiments disclosed herein where multiple beams are used.

FIGS. 9-11B show an alternate design in which multiple exit ports dispersed circumferentially around the tool emit beams. As illustrated in FIGS. 9 and 10, four exit ports 38a,38b,38c,38d emit beams 42a,42b,42c,42d to create optical targeting markers 40a,40b,40c,40d. The light from all exit ports may be generated by a single light source, for example within the optic unit described above, either on the tool or remotely. Alternatively, multiple light sources may be provided, each supplying light to one or more of the exit ports. If so, conventional light guides can be used to split the generated light into different optical fibers leading to each exit port, as desired.

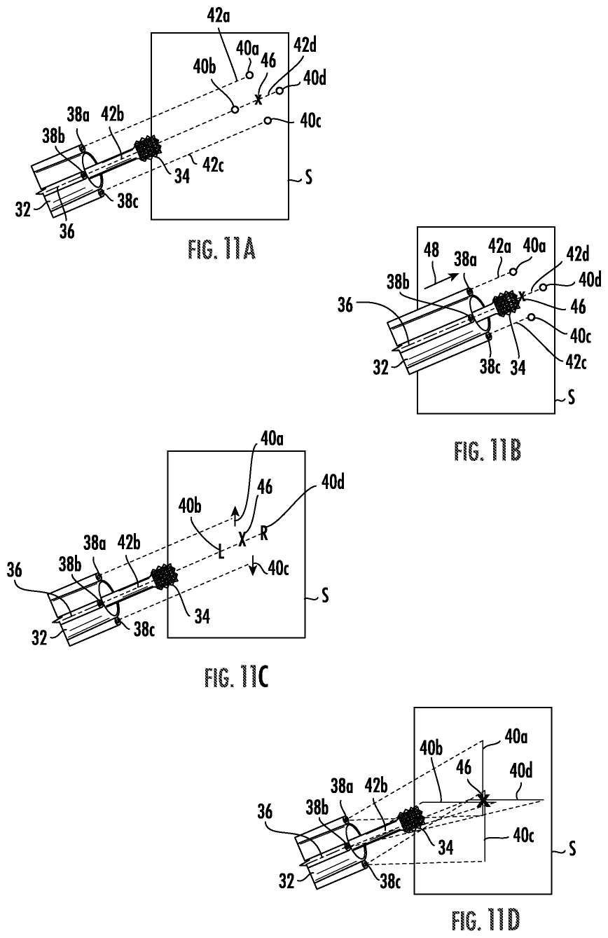

The beams generated by the device of FIG. 9 extend parallel to central axis 36 and to operative direction 48 (as shown in FIG. 11B), although as noted above the beams may be oriented in other relative orientations. Each beam projects to an equal distance 44a,44b,44c,44d (see FIG. 10) away from desired contact point 46, although the distances need not be equal. FIGS. 11A and 11B show that the parallel beams 42a-42d create markers 40a-40d that remain equally spaced from contact point 46 as the tool is moved in operative direction 48 parallel to central axis 36.

Although four such beams arranged 90 degrees apart are shown in FIGS. 9-11B, it should be understood that two or more beams may be employed arranged around point 46 as desired, in equal or unequal distances and/or circumferential arrangement.

FIG. 11C shows a light modification to the device of FIGS. 9-11B, wherein one or more of the four optical targeting markers 40a-40d may be something other than a dot or a line. As shown, the markers in FIG. 11C comprise indicia to provide the surgeon information as to orientation of tool 20 relative to target 46 (X). Marker 40d is a letter R (for right), marker 40b is a letter L (for left), and markers 40a and 40c are arrows (for up and down, respectively). When the tool is oriented (by the surgeon or robot) so that the marker 40a (up arrow) is pointing upward, use of such indicia on one or more of the markers can provide additional feedback that can help with manipulation of the tool, either directly or robotically though a user-operated interface (for example, with similar indicia on it input device(s)). Any symbol, indicia, design, etc., may be created to allow one or more of the optical targeting markers to provide additional information to the surgeon. Such information may be more important where manipulation portion is not symmetrical relative to the tool, or where orientation of the tool within a surgical site or during a particular surgical step is critical.

FIG. 11D shows another variation in which four optical targeting markers 40a-40d together form two lines crossing at target site 46. Each of the exit ports 38a-38d is configured to emit a beam 42a-42d in a widening line, and the beams may all meet and overlap at the target site 46 along axis 36. It would be possible to create such a cross with only two such exit ports spaced 90 degrees apart, but they would have to be arranged so that no shadowing is caused by the manipulation member 34. As tool 20 is moved closer to the target site 46, the cross formed by optical targeting markers 40a-40d will become smaller, thereby providing the surgeon with both alignment and distance feedback.

FIGS. 12A and 12B show another variation where multiple exit ports emit non-parallel beams that cross over the target area. As shown, two exit ports 38a,38b are provided on opposite sides of center axis 36. Beams 42a,42b are emitted and cross just in front of manipulation member 34. When the tool is in the position of FIG. 11A, optical targeting markers 40a,40b are spaced from contact point 46 by distance 44. When the tool is moved in the operating direction 48, optical targeting markers 40a,40b converge on point 46, thereby assisting the surgeon with alignment and providing distance to target information as distance 44 is reduced. It should be understood that more than two of such exit ports could be employed, arrayed as desired, evenly or unevenly, in one or more course at different angles or spacings, around center axis 36.

FIGS. 13A and 13B show a show an alternate design in which one or more exit ports includes optics, lensing, etc., which can emit a beam split into at least two portions so as to extend from the exit port in indifferent directions. Splitting of beams can be more spatially and commercially efficient than providing one exit port per beam, at least in certain applications. As shown, two exit ports 38a,38b emit four beams 42a1,42a2,42b1,42b2 to create optical targeting markers 40a1,40a2,40b1,40b2. Beams 42a1 and 42b1 extend forward, parallel to central axis 36, and beams 42a2 and 42b2 extend (aligned with each other) perpendicular to central axis 36. The four beams and markers can assist the surgeon in identifying parts of the surgical cavity apart from the target area as the tool is manipulated during a procedure. There may be one or more splitters, which may each split the light into two or more beams. The beams may also be aimed in different directions, parallel, non-parallel, perpendicular, skewed, etc. The light from each exit port may be generated by a single light source, for example within the optical unit described above, either on the tool or remotely. Alternatively, multiple light sources may be provided, each supplying light to one or more of the exit ports. If so, conventional light guides can be used to split the generated light into different optical fibers leading to each exit port, as desired. Thus, use of beam splitting provides further options for creation and direction of the optical targeting markers.

FIGS. 14A and 14B show a show an alternate design of a manipulation portion 130 of a tool 120. Manipulation portion 130 includes a shield 131 to prevent contact with portions of the surgical site but to also allow contact with other portions of the surgical site as the tool is moved and used. Such a shield can have different arrangements and orientations, depending on the procedures. As shown, shield 131 covers a bottom side 134a and distal end 134b of manipulation member 134 of manipulation portion 130.

The presence of shield 131 allows at least one exit port to be placed therein to project an optical targeting marker therefrom. As shown, two exit ports 138a,138b are located at or adjacent distal end 131a of shield, and two exit ports 138c,138d are located in a proximal area 131b. The four exit ports are located laterally around manipulation member 134 and emit four beams 142a,142b,142c,142d to create optical targeting markers 140a,140b,140c,140d. The beams extend parallel to operative direction 148 (leading to center target area contact point 146), the beams and the operative direction herein being perpendicular to central axis 136.

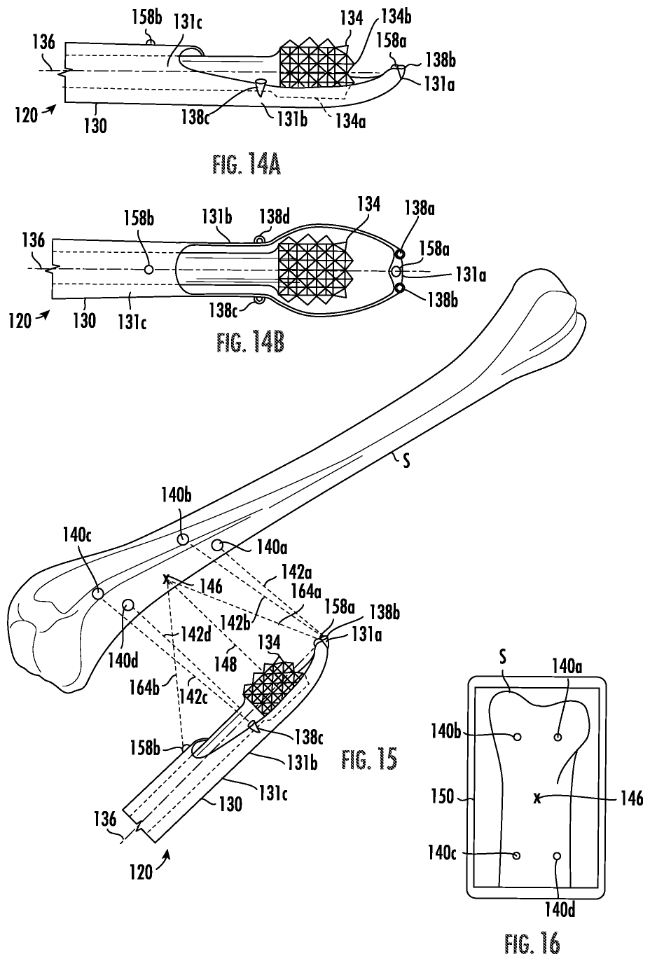

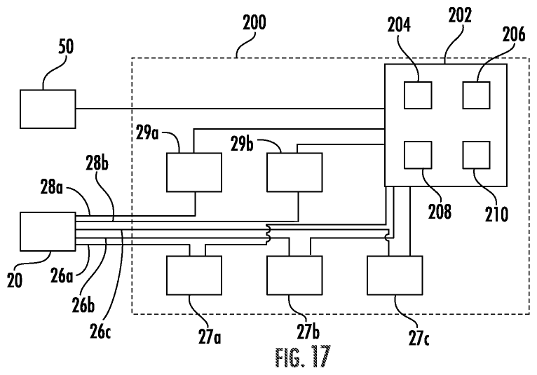

The surgical site and optical targeting markers may be visualized directly (FIG. 15) or on a display screen 150 (FIG. 16, through use of optical receivers on tool 120—within or separate from the exit ports) or separately provided. Thus, if desired, one or more than one optical receivers 158a,158b may be provided on tool 120 for imaging the surgical site and the optical targeting markers 140a-140d. As shown, optical receiver 158a is at distal end 131a of shield and another optical receiver 158b is adjacent proximal end 131c of shield portion 130. Each optical receiver may provide its own view, or the views provided by the optical receivers may be combined using computer processing to create a single view, two dimensionally or in stereopsis. As shown, two receivers 158a,158b are provided, located axially on top of tool 120 pointing toward the operative direction, but two or more receivers could be distributed differently, and could point outwardly in more directions to provide an up to 360-degree view of the entire wound cavity, if desired.

The optical receivers may be connected, for example, by optical fibers and waveguides to an optical unit having one or more CCDs, such as unit 60 noted above. If so, by use of triangulation of the location of a given point in the surgical site using two optical receivers of known position on the tool (for example by identifying pixels of CCDs impacted by an item viewed from two locations), a distance to the given point can be determined. Thus, distance to desired contact point 146 or one of the optical targeting markers 140a-140d can be determined using two optical receivers 158a,158b. The distance information can be provided to the surgeon indifferent ways, e.g., by screen readout, by augmented reality on a screen such as screen 150, etc.

FIG. 17 shows a schematic representation of elements of the tools described herein, along with interconnection to other elements within a surgical system. As noted, the tools noted herein can be of many varieties, so the systems for controlling them could vary accordingly in conventional ways as understood by those skilled in the art.

As illustrated and as described above, tool 20 may be connected to cables 26a-26c and conduits 28a,28b. The cables may be connected for example to elements within system 200, such as a power module 27a, a control module 27b, and an optical communication module 27c (which may comprise or include features of optical unit 60, as described above). The conduits may be connected to a surgical fluid supply 29a and suctioning device 29b for removing the fluid along with any material removed from the patient during the procedure. Each of the modules and fluid supply/removal are functionally controlled and/or powered by one or more controllers 202 (one is shown), within system 200. As schematically illustrated for simplicity, controller 202 may include or be connected to a central processing unit 204, a storage memory 206, programming and other stored data 208, and at least one input-output device 210 for turning on and controlling features and functions of tool 20, including the modules 27a-27c, the fluid supply removal 29a,29b, and the display 50.

It should be understood that several separate controllers may actually be supplied for controlling different portions of the tool and the overall surgical system. The controller or controllers need not be local to the tool; they may be at least partially remote (on-site) or remote (offsite), connected by conventional wired or wireless connections. The input-output device 210 may include manipulatable input devices such as buttons, triggers, switches, keyboards, keypads, touchscreens, microphones, etc., on the tool itself as well as on other devices within the system for controlling elements of the system. For output, items such as screens, lights, indicators, speakers, etc., can be provided to give the user feedback as to status of the tool, its components, or the procedure. One or more conventional power supplies (not shown) may be provided for powering the tool, the controller, and all other elements shown in FIG. 17.

In use, surgical tools such as those discussed above having targeting and/or guidance features may be beneficially employed in surgeries characterized as “delicate” in terms of the tissue to be manipulated being in close vicinity of tissue which must not be disturbed or damaged, e.g., nerves, blood vessels, etc. Also, such devices may be beneficial for surgeries in which tissue to be manipulated is hard to reach and/or not directly observable, e.g., surgery on the vertebrae with an entry point on the back but with the surgery to be performed on the ventral side.

In using a such a tool during a surgery, for example the tool of FIG. 14A/B, the surgeon would first turn on a perform a function/diagnostic check of the tool and connected system to ensure all is in working order and connected properly. The manipulation member and projecting devices would then be switched off. The tool would be inserted in a wound cavity shield first to prevent the working side from getting stuck or undesirably rupturing tissue, e.g., in case of a tool with a grinder head. The manipulation member of the tool as well as the projecting devices could be turned off during the approach phase and only the camera/cameras would be switched on from the start. The projecting device/devices could be switched on to create the optical targeting marker(s) after the tool is inserted into the wound, so as to ensure that the beams would not accidently hit the eye of one of the surgical team. The tool is then guided by both feel and observation on the screen (as in FIGS. 14A/B-15, or through direct visual observation if that were being used) to the rough vicinity of the desired contact area within the surgical site.

Upon approaching the surgical site and the to-be-avoided tissue, the approach would be guided mainly by the tool's camera/cameras using the targeting markers and any distance information provided on the screen. Thus, it is known how far or how close the tool and the manipulation member of the tool are from the targeted surgical site and the to-be-avoided areas.

When the surgical site is reached and in view from the tool, the tool would be fine aligned, especially the manipulation member towards the surgical site. The fine alignment can be checked by any distance information provided on the screen by electrooptical distance determination as well as information about the distances within the wound cavity which can be derived. When multiple target markers are used, which outline/edge the area of the working surface and this outline coincided with the area intended for surgery while avoiding any areas which are sensitive, the tool's working surface (manipulation member) would be switched on.

The tool, with the manipulation member switched on, is then moved carefully towards the surgical site, ensuring that the proper alignment is not lost during the approach and the main part of the surgical procedure begins, e.g., removal of tissue. Alignment information feedback is provided to the surgeon by the optical targeting markers, makers on the tool, and such during this movement so that desired alignment is maintained through contact. After a short contact, the tool may be moved back to observe the procedure's progress and the tool may be realigned, if desired for further contact with the target area or an adjacent target area, and then moved into contact. Such aligning and contacting may be repeated as many times as necessary until the procedure is concluded, for example, when the goal of the tissue manipulation is reached and confirmed by observation and/or measurement by electrooptical distance determination, either by reference to certain landmarks in the wound and/or the optical targeting markers and/or determination from provided information/images.

In the last phase of the surgical procedure, the tool would be retracted from the surgical site with the manipulation member switched off, essentially opposite from the steps in the first approach/insertion phase. During the extraction it is as paramount as during the insertion that the surgeon avoids contact with sensitive tissue and unwanted damage to tissue.

The surgical procedure can be used with image-guided-surgical methods, e.g., to complement the information form the targeting and imaging, especially the 1st-person view, with the positioning and alignment information provided by the 3rd-person information from image-guided-systems, e.g., X-ray, ultrasound, CT, MRI, positron emission tomography. This information can either be displayed in addition to the images from the tool, images from an endoscope observing the tool, on one split screen, multiple screens or combined with a virtual display, a virtual reality image and/or an augmented reality image. Alternatively, and additionally, the different views displayed may be chosen and switched between by the surgeon, depending on the preferred viewpoint for each phase of the surgical procedure.

The surgical procedure using the tools disclosed above can also be performed by a robot administered/controlled by a surgeon, who can choose the image/viewpoint before and during the procedure according to preference. Other support devices, like N-localizers, which help to improve precision and quality of the procedure, may also be employed.

Thus, the disclosed subject matter provides an easy to use and user-friendly device and system for assisting in surgical procedures by providing at least one optical targeting marker projected from a surgical tool. This disclosure is applicable to many different surgical tools and procedures, and the optical targeting markers can have many forms. Therefore, the disclosed concepts are not intended to be constrained to the application of any particular tool or procedure, or any number of or orientation of, optical targeting marker(s).

While preferred embodiments of the invention have been fully described above, it is to be understood that any and all equivalent realizations of the present invention are included within the scope and spirit thereof. Thus, the embodiments depicted are presented by way of example only and are not intended as limitations upon the present invention. Thus, while particular embodiments of the invention have been described and shown, it will be understood by those of ordinary skill in this art that the present invention is not limited thereto since many modifications can be made. Therefore, it is contemplated that any and all such embodiments are included in the present invention as may fall within the literal or equivalent scope of the appended claims.

1. A powered surgical tool for a user to manipulate a desired tissue in surgical site within a patient, the powered surgical tool comprising:
a body including a main portion and a tissue manipulation portion, the tissue manipulation portion being powerable and having a manipulation member for manipulating the desired tissue, the body and the tissue manipulation portion being configured together so as to define an operative direction of the manipulation member relative to the surgical site, wherein the tissue manipulation member is powered and aligned in the operative direction to manipulate the desired tissue disposed along the operative direction; and
a projecting device with an exit port, the projecting device being powerable to emit an optical targeting marker from the exit port in a targeting direction onto the surgical site, the exit port being located on the body so that the targeting direction is operationally related to the operative direction, wherein the optical targeting marker is visible to the user for providing information about at least one of a location and an orientation of the manipulation member within the surgical site and for providing information about at least one of a location and an orientation of the operative direction within the surgical site.

2. The powered surgical tool of claim 1, wherein the operative direction and the targeting direction are parallel.

3. The powered surgical tool of claim 2, wherein the body includes a central portion and a distal end extending in a distal direction from the central portion, and wherein the operative direction and the targeting direction extend in the distal direction.

4. The powered surgical tool of claim 2, wherein the body includes a central portion and a distal end extending in a distal direction from the central portion, and wherein the operative direction and the targeting direction extend in a lateral direction angled from the distal direction.

5. The powered surgical tool of claim 4, wherein the lateral direction is perpendicular to the distal direction.

6. The powered surgical tool of claim 1, wherein the projecting device includes an electrically-powered light source and the exit port includes a lens for directing light from the electrically-powered light source in a beam in a targeting direction to form the optical targeting marker.

7. The powered surgical tool of claim 6, further including an optical unit remote from the tool, the electrically-powered light source being located in the optical unit.

8. The powered surgical tool of claim 7, wherein the optical unit further includes a camera for receiving an image obtained of the surgical site.

9. The powered surgical tool of claim 1, wherein the exit port emits a light beam having parallel rays so that a size of the optical targeting marker is substantially unchanged regardless of a distance between the exit port and the optical targeting marker within the surgical site.

10. The powered surgical tool of claim 9, wherein the optical targeting marker includes at least one point.

11. The powered surgical tool of claim 9, wherein the optical targeting marker includes at least one line.

12. The powered surgical tool of claim 9, wherein the optical targeting marker is sized relative to the manipulation member so as to indicate the area within the surgical site that would be contacted by the manipulation member if moved in the operative direction to contact the desired tissue.

13. The powered surgical tool of claim 1, wherein the exit port emits a light beam having non-parallel rays centered around the targeting direction so that a size of the optical targeting marker changes depending on a distance between the exit port and the optical targeting marker within the surgical site.

14. The powered surgical tool of claim 1, wherein the exit port emits a light beam, and the tool includes a detector for electrooptical distancing to determine a distance between the exit port and the optical targeting marker within the surgical site.

15. The powered surgical tool of claim 14, wherein the detector includes a camera within or connected to an optical receiver.

16. The powered surgical tool of claim 1, wherein the projecting device is configured to emit at least two light beams from at least one exit port, the optical targeting marker including distinct portions created by each of the at least two light beams.

17. The powered surgical tool of claim 16, wherein two of the distinct portions are of different wavelengths in a visible spectrum.

18. The powered surgical tool of claim 16, wherein the distinct portions are spaced relative to the manipulation member so as to indicate the area within the surgical site that would be manipulated by the manipulation member if moved in the operative direction to contact the desired tissue.

19. The powered surgical tool of claim 16, wherein the at least one exit port is a single exit port configured to split the light into the at least two beams.

20. The powered surgical tool of claim 16, wherein the at least one exit port includes a plurality of exit ports, each of the exit ports being configured to emit one of the at least two beams.

21. The powered surgical tool of claim 1, further including at least one imaging device for obtaining an image of the surgical site.

22. The powered surgical tool of claim 21, wherein the at least one imaging device uses an optical receiver attached to the body for obtaining the image of the surgical site.

23. The powered surgical tool of claim 22, wherein the imaging device includes a camera for receiving the image from the optical receiver.

24. The powered surgical tool of claim 23, wherein the camera is located in an optical unit remote from the tool.

25. The powered surgical tool of claim 24, wherein the projecting device includes an electrically-powered light source and the exit port includes a lens for directing light from the electrically-powered light source in a beam in a targeting direction to form the optical targeting marker, the electrically powered light source being located in the optical unit.

26. The powered surgical tool of claim 22, including a plurality of the exit ports and a plurality of the optical receivers distributed about the tool.

27. The powered surgical tool of claim 22, wherein the at least one imaging device uses a plurality of the optical receivers, the images obtained by the optical receivers devices being complimentary to each other.

28. The powered surgical tool of claim 27, wherein the optical receivers are spaced from each other on the body.

29. The powered surgical tool of claim 21, wherein one of the imaging devices obtains an image including the optical targeting marker along the operative direction, and another of the imaging devices obtains an image directed differently from the operative direction.

30. The powered surgical tool of claim 21, wherein the at least one imaging device includes an optical receiver, and wherein the optical receiver is also the exit port of the projecting device.

31. The powered surgical tool of claim 1, further including positioning markings distributed on the body for identifying an orientation of the body by detecting positions of the positioning markings.

32. The powered surgical tool of claim 1, further including a display device for displaying an image providing the information about the at least one of the orientation of and the location of the manipulation member within the surgical site and for providing the information about the at least one of the location and the orientation of the operative direction within the surgical site.

33. The powered surgical tool of claim 32, wherein the image is a visual image of the surgical site.

34. The powered surgical tool of claim 33, wherein the image includes the optical targeting marker.

35. The powered surgical tool of claim 32, wherein the image includes a representation of the location and the orientation of the manipulation member relative to the desired tissue.

36. The powered surgical tool of claim 32, wherein the image is an augmented reality image.

37. The powered surgical tool of claim 1, wherein the manipulation member is one of a laser element, an ultrasonic element, an oscillating element, a rotary element, a thermal element, a radiofrequency element, an electrical element, an electromagnetic element, a magnetic element, a radiological element, a chemical element.

38. The powered surgical tool of claim 1, wherein the body includes a handle and a shield, the shield being located adjacent the manipulation member of the tissue manipulation portion and configured to at least partially surround a first part of the manipulation member so that the first part is precluded from contact with the surgical site, the shield configured so that a second part of the manipulation member is exposed for use in manipulating the desired tissue within the surgical site.

39. The powered surgical tool of claim 38, wherein the exit port is located on the shield.

40. The powered surgical tool of claim 39, wherein at least a distal portion of the shield is located on the body distally relative to the manipulation member and the handle, and the exit port is located on the distal portion of the shield.

41. The powered surgical tool of claim 38, wherein the projecting device is configured with two of the exit ports, one of the exit ports being located on the shield and another of the exit ports being located on the body spaced from the shield.

42. The powered surgical tool of claim 38, further including an imaging device with at least two optical receivers attached to the body for obtaining the image of the surgical site, at least one of the optical receivers being located on the shield and at least another of the optical receivers being located on the body spaced from the shield.
